(19) United States
(12) Patent Application Publication
(10) Pub. No.: US 2022/0380320 A1
(43) Pub. Date: Dec. 1, 2022

(54) PHARMACEUTICAL COMPOSITION FOR TREATING OR PREVENTING MIDDLE EAST RESPIRATORY SYNDROME

(71) Applicant: GYEONGGIDO BUSINESS & SCIENCE ACCELERATOR, Suwon-si, Gyeonggi-do (KR)

(72) Inventors: Jin-Mo KU, Suwon-si, Gyeonggi-do (KR); Kwi-Wan JEONG, Yongin-si, Gyeonggi-do (KR); Seung-Young JUNG, Suwon-si, Gyeonggi-do (KR)

(73) Assignee: GYEONGGIDO BUSINESS & SCIENCE ACCELERATOR, Suwon-si, Gyeonggi-do (KR)

(21) Appl. No.: 17/427,778

(22) PCT Filed: Feb. 3, 2020
(86) PCT No.: PCT/KR2020/001600

§ 371 Date: Jul. 28, 2022

(30) Foreign Application Priority Data
Feb. 1, 2019 (KR) 10-2019-0013936

(51) Int. Cl. C07D 239/10; A61K 45/06; C07D 409/06

(52) U.S. Cl. CPC C07D 239/10; A61K 45/06; C07D 409/06

(57) ABSTRACT

The present invention relates to an amide derivative compound or a pharmaceutically acceptable salt thereof, which can be beneficially used in the treatment or prevention of Middle East Respiratory Syndrome (MERS). In addition, the present invention relates to a pharmaceutical composition for treating or preventing MERS, comprising said compound or a pharmaceutically acceptable salt thereof.

### TECHNICAL FIELD

The present invention relates to an amide derivative compound or a pharmaceutically acceptable salt thereof, which can be beneficially used in the treatment or prevention of Middle East Respiratory Syndrome (MERS). In addition, the present invention relates to a pharmaceutical composition for treating or preventing MERS, comprising said compound or pharmaceutically acceptable salt thereof.

### BACKGROUND ART

Middle East Respiratory Syndrome, also referred to as MERS, is a severe acute respiratory disease caused by a new type of coronavirus infection, which has not been found in humans in the past. Symptoms such as fever, cough, dyspnea, and acute renal failure may develop after an incubation period of 2 to 14 days. Currently, the source and route of MERS infection have not been clearly identified, and a standardized treatment has not been established worldwide.

According to the European Center for Disease Control and Prevention (ECDC), as of May 29, 2015, the first case of MERS occurred in 2012 and most of the patients and deaths related to the infection were found in the Middle East. In South Korea, after the first confirmed case of MERS, found from a man who entered Korea from Bahrain in May 2015, it became a country with the second highest number of MERS cases in the world after Saudi Arabia.

On 15 Jun. 2015, MERS antiviral treatment guidelines were published in South Korea, recommending the combined administration of ribavirin, interferon-α2a, and lopinavir/ritonavir as antiviral treatment. The MERS antiviral treatment guidelines were published based on clinical research papers provided by the International Severe Acute Respiratory and Emerging Infection Consortium (ISARIC), a group of Public Health England (PHE).

However, lopinavir has only been approved for the treatment of HIV-1 infection, and its effectiveness against MERS has not been proven yet. Also, there are studies that report lopinavir/ritonavir had no activity against MERS-CoV (Middle East Respiratory Syndrome-Coronavirus) in vitro. Therefore, it is understood that the efficacy of lopinavir/ritonavir for the treatment of MERS has not been specifically demonstrated and that it was used as a temporary expedient for a concomitant administration in the absence of a standard treatment.

In 2015, the fatality rate of MERS reached 20.4% in South Korea, but no treatment has been specifically proven to be effective. Therefore, there is an urgent need for a therapeutic agent for the treatment or prevention of Middle East Respiratory Syndrome (MERS).

### TECHNICAL PROBLEM

The present invention is directed to a compound or a pharmaceutically acceptable salt thereof that can be beneficially used in the treatment or prevention of Middle East Respiratory Syndrome (MERS), and a pharmaceutical composition comprising the same.

### TECHNICAL SOLUTION

The present inventors have developed a compound with high antiviral activity against MERS-CoV with low cytotoxicity to normal cells by screening hundreds of thousands of compounds provided from the Gyeonggi Bio Center, located in Gyeonggido Business & Science Accelerator.

As a result, it was confirmed that particular amide derivative compounds or pharmaceutically acceptable salts thereof exhibit desired effects, and the compounds can be represented as Formula 1:

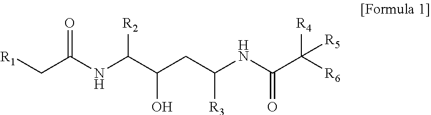

### Wherein

R₁ is —OR₇ or —SR₇,
   where R₇ is C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl or C3-10 heterocycloalkylalkyl, substituted or unsubstituted by one or more substituent selected among halogen, C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl and C3-10 heterocycloalkylalkyl;
Each R₂ and R₃ are independently hydrogen, C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl, or C3-10 heterocycloalkylalkyl;
R₅ is

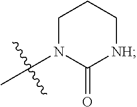 [SMILES: *N1CCCNC1=O]

in case that R₄ is a hydrogen atom,
   R₆ is C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl, or C3-10 heterocycloalkylalkyl, substituted or unsubstituted by one or more substituent selected among halogen, C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl, C3-10 heterocycloalkylalkyl, —CF₃ and —CN;
in case that R₄ is C1-C6 alkyl,
   R₆ is C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl, or C3-10 heterocycloalkylalkyl;
R₄ and R₆ can be combined to form C6-C12 aryl, C3-C12 cycloalkyl, C3-10 heteroaryl or C3-10 heterocycloalkyl.

In one embodiment, each substituent R₂ and R₃ of Formula 1 are independently C6-C12 aryl, C6-C12 arylalkyl, C3-10 heteroaryl or C3-10 heteroarylalkyl. Substituents R₂ and R₃ of Formula 1 may be the same as or different from each other. The substituents R2 and R3 may be substituted or unsubstituted by one or more substituents selected among halogen, C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3 -10 heteroarylalkyl, C3-10 heterocycloalkyl, C3-10 heterocycloalkylalkyl, —CF3 and —CN.

In one embodiment, when R₄ of Formula 1 is a hydrogen atom, R₆ is C6-C12 aryl, C6-C12 arylalkyl, C3-10 heteroaryl or C3-10 heteroarylalkyl. R₆ may be substituted or unsubstituted by one or more substituent selected among halogen, C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl, C3-10 heterocycloalkylalkyl, —CF₃ and —CN.

In another embodiment, when R₄ of Formula 1 is C1-C6 alkyl, R₆ is C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl or C3-10 heterocycloalkylalkyl. R₆ may be substituted or unsubstituted by one or more substituent selected from the group consisting of: halogen, C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl, C3-10 heterocycloalkylalkyl, —CF₃ and —CN.

In a more preferred embodiment, the compound of Formula 1 according to the present invention is selected from the group consisting of:
(S)-N-((2S,4S,5S)-5-(2-(2,6-dimethylphenoxy)acetamido)-4-hydroxy-1,6-diphenylhexane-2-yl)-2-(2-oxotetrahydropyrimidin-1(2H)-yl)-3-phenylpropanamide;
(S)-N-((2S,4S,5S)-5-(2-(2,6-dimethylphenoxy)acetamido)-4-hydroxy-1,6-diphenylhexane-2-yl)-2,3-dimethyl-2-(2-oxotetrahydropyrimidin-1(2H)-yl)butanamide;
(R)-N-((2S,4S,5S)-5-(2-(2,6-dimethylphenoxy)acetamido)-4-hydroxy-1,6-diphenylhexane-2-yl)-2,3-dimethyl-2-(2-oxotetrahydropyrimidin-1(2H)-yl)butanamide;
N-((2S,4S,5S)-5-(2-(2,6-dimethylphenoxy)acetamido)-4-hydroxy-1,6-diphenylhexan-2-yl)-1-(2-oxotetrahydropyrimidin-1(2H)-yl)cyclopentanecarboxamide;
(S)-N-((2S,4S,5S)-5-(2-(2,6-dimethylphenoxy)acetamido)-4-hydroxy-1,6-diphenylhexane-2-yl)-2-(2-oxotetrahydropyrimidin-1(2H)-yl)-3-(m-tolyl)propanamide;
(S)-N-((2S,4S,5S)-5-(2-(2,6-dimethylphenoxy)acetamido)-4-hydroxy-1,6-diphenylhexane-2-yl)-2-(2-oxotetrahydropyrimidin-1(2H)-yl)-3-(p-tolyl)propanamide;
(S)-N-((2S,4S,5S)-5-(2-(2,6-dimethylphenoxy)acetamido)-4-hydroxy-1,6-diphenylhexane-2-yl)-3-(4-fluorophenyl)-2-(2-oxotetrahydropyrimidin-1(2H)-yl)propanamide;
(S)-N-((2S,4S,5S)-5-(2-(2,6-dimethylphenoxy)acetamido)-4-hydroxy-1,6-diphenylhexane-2-yl)-2-(2-oxotetrahydropyrimidin-1(2H)-yl)-3-(4-(trifluoromethyl)phenyl)propanamide;
(S)-3-(4-cyanophenyl)-N-((2S,4S,5S)-5-(2-(2,6-dimethylphenoxy)acetamido)-4-hydroxy-1,6-diphenylhexan-2-yl)-2-(2-oxotetrahydropyrimidin-1(2H)yl)propanamide;
(S)-N-((2S,4S,5S)-5-(2-(2,6-dimethylphenoxy)acetamido)-4-hydroxy-1,6-diphenylhexane-2-yl)-2-(2-oxotetrahydropyrimidin-1(2H)yl)-3-(thiophen-2-yl)propanamide;
(S)-N-((2S,4S,5S)-5-(2-(2,6-dimethylphenoxy)acetamido)-4-hydroxy-1,6-diphenylhexane-2-yl)-2-(2-oxotetrahydropyrimidin-1(2H)yl)-3-(thiazol-5-yl)propanamide;
(S)-3-cyclopentyl-N-((2S,4S,5S)-5-(2-(2,6-dimethylphenoxy)acetamido)-4-hydroxy-1,6-diphenylhexan-2-yl)-2-(2-oxotetrahydropyrimidin-1(2H)yl)propanamide; and
(S)-2-cyclopentyl-N-((2S,4S,5S)-5-(2-(2,6-dimethylphenoxy)acetamido)-4-hydroxy-1,6-diphenylhexan-2-yl)-2-(2-oxotetrahydropyrimidin-1(2H)-yl)acetamide.

The terms “alkyl, alkenyl, alkynyl” used herein include both linear or branched forms.

The term “halogen” used herein refers to fluorine, chlorine, bromine, or iodine.

The term “hetero” in “heteroaryl, heteroarylalkyl, heterocycloalkyl, heterocycloalkylalkyl” used herein refers to atoms selected from oxygen, nitrogen and sulfur included in the ring.

The terms “arylalkyl, cycloalkylalkyl, heteroarylalkyl, heterocycloalkylalkyl” used herein refer to an aryl ring connected to an alkyl group, a cycloalkyl ring connected to an alkyl group, a heteroaryl ring connected to an alkyl group, and a heterocycloalkyl ring connected to an alkyl group, respectively.

The term “independently” used herein means that two or more substituents may be individually defined and may be different from or the same as each other.

The compound of Formula 1 may have an asymmetric carbon center forming isomers such as R or S isomers, racemates, diastereomers, or a mixture thereof; all of which are included in the scope of the present invention.

In another aspect, the present invention provides a pharmaceutical composition comprising a compound represented by Formula 1 or a pharmaceutically acceptable salt thereof. The pharmaceutical composition may be used for the treatment or prevention of Middle East Respiratory Syndrome.

In addition, the present invention provides a method for treating or preventing Middle East Respiratory Syndrome, comprising administering a pharmaceutical composition comprising the compound of Formula 1 or a pharmaceutically acceptable salt thereof as an active ingredient to a subject (e.g. an animal, preferably a mammal, more preferably a human).

The term “prevention” used herein refers to all actions that inhibit, delay or prevent the onset of Middle East Respiratory Syndrome by administering a pharmaceutical composition to a subject at high risk of Middle East Respiratory Syndrome. In addition, the term “treatment” used herein refers to all actions that improve, reverse, cure or improve symptoms by administering the pharmaceutical composition to a subject suffering from Middle East Respiratory Syndrome or suspected of having Middle East Respiratory Syndrome.

The content of the compound of Formula 1 or a pharmaceutically acceptable salt thereof comprised in the pharmaceutical composition of the present invention can be appropriately adjusted according to the symptoms of the disease, the degree of progression of the symptoms, the condition of the patient, and the like. For example, based on the total weight of the pharmaceutical composition, the content of the compound of Formula 1 or a pharmaceutically acceptable salt thereof according to the present invention may be contained in an amount of 0.0001 to 99.9%, 0.1 to 90%, 1 to 80%, 1 to 70%, 1 to 60%, or 1 to 50% by weight.

The term “pharmaceutically acceptable salt” used herein refers to a compound that can be prepared by a conventional method in the art, for example, hydrochloric acid, hydrogen bromide, sulfuric acid, sodium hydrogen sulfate, phosphoric acid, carbonic acid, etc. salts with inorganic acids of formic acid, acetic acid, oxalic acid, benzoic acid, citric acid, tartaric acid, gluconic acid, gestisic acid, fumaric acid, lactobionic acid, salicylic acid; or use organic acids such as acetylsalicylic acid (aspirin) to form acceptable salts of the acids thereof; or react with an alkali metal ion such as sodium or potassium to form a metal salt thereof; or react with an ammonium ion to form another pharmaceutically acceptable salt.

The pharmaceutical composition of the present invention may further comprise other active ingredients, such as one or more antiviral agents, if necessary, together with the compound of Formula 1 or a pharmaceutically acceptable salt thereof.

In addition, the pharmaceutical composition according to the present invention may further comprise a pharmaceutically acceptable carrier, diluent, or excipient. The term “pharmaceutically acceptable carrier or diluent” used herein refers to a carrier or diluent that does not cause significant irritation to a subject and does not alter or decrease the biological activity and properties of a compound or salt to be administered. In addition, the term “pharmaceutically acceptable excipient” used herein refers to inert substances added to a pharmaceutical composition to further facilitate administration of the compound or salt of the present invention. Examples of such excipients include, but are not limited to, calcium carbonate, calcium phosphate, various types of sugars and starches, cellulose derivatives, gelatin, vegetable oils and polyethylene glycols.

The pharmaceutical composition according to the present invention may be administered to a subject by various routes. For example, but not limited to oral, rectal, or intravenous, topical or parenteral routes of administration.

The composition according to the present invention may be formulated as a formulation for oral administration or a formulation for parenteral administration without being limited to a specific formulation. In this case, the active ingredient according to the present invention may be contained in unit doses in the formulation or may be contained in aliquots. When formulating the composition according to the present invention, diluents or excipients commonly used in the art such as conventional fillers, extenders, binders, wetting agents, surfactants may be additionally used. Solid preparations for oral administration may include, for example, tablets, pills, powders, granules, capsules, and these solid preparations may comprise one or more excipients such as starch, calcium carbonate, sucrose, lactose, or gelatin and the like in addition to the active ingredient.

Liquid formulations for oral administration may include, for example, suspensions, internal solutions, emulsions and syrups, and these liquid formulations may include commonly used simple diluents such as water and liquid paraffin, and various excipients, such as wetting agents, sweeteners, fragrances, preservatives, and the like. Formulations for parenteral administration may include, for example, sterile aqueous solutions, non-aqueous agents, suspensions, emulsions, lyophilized formulations, and suppositories. Materials such as propylene glycol, polyethylene glycol, vegetable oils such as olive oil, and injectable esters such as ethyl oleate may be used as a suspending agent.

The pharmaceutical composition of the present invention is administered to a subject in a pharmaceutically effective amount. The term “pharmaceutically effective amount” used herein refers to an amount sufficient to treat a disease with a reasonable benefit/risk ratio applicable to medical treatment, and the effective dose level can be determined by subject type and severity including factors such as age, sex, activity of the drug, sensitivity to the drug, administration time, administration route, excretion rate, duration of treatment, usage of concurrent drugs, and other factors well known in the medical field.

The pharmaceutical composition of the present invention may be administered as an individual therapeutic agent or may be administered in combination with other therapeutic agents, in which the composition can be administered sequentially or simultaneously with conventional therapeutic agents. In addition, the composition may be administered in single and multiple doses. In consideration the above factors, it is important to administer the minimum amount that can obtain the maximum effect without side effects, which can be easily determined by a person skilled in the art.

The compound according to the present invention or a pharmaceutically acceptable salt thereof exhibits excellent antiviral activity selectively against MERS-CoV while having low cytotoxicity to host cells.

The compound according to the present invention or a pharmaceutically acceptable salt thereof significantly inhibits the proliferation and replication of MERS-CoV, thereby demonstrating the effectiveness and safety of being used as an active ingredient in a pharmaceutical composition. The present invention can provide a pharmaceutical composition for the treatment or prevention of Middle East Respiratory Syndrome.

In particular, the pharmaceutical composition according to the present invention is advantageous in various aspects such as exhibiting high antiviral effect even in low concentration, enabling treatment in early stage of infection or during the incubation period, and showing cytotoxicity only against MERS-CoV and not against normal cells.

Moreover, a single administration of the pharmaceutical composition according to the present invention can sufficiently achieve the desired therapeutic or preventive effect of Middle East Respiratory Syndrome, which eliminates potential problems from taking combined administrations such as increased cost and patient discomfort.

### EXAMPLES

Hereinafter, preferred examples are presented to help the understanding of the present invention. However, the following examples are provided for easier understanding of the present invention, and the contents of the present invention are not limited by the following examples. Those of ordinary skill in the art may make various modifications to the present invention within the scope not departing from the inventive concept.

### Example 1

### Preparation of Compounds of Formula 1

In the following, examples of preparation of compounds of Formula 1 according to the present disclosure are described below. Preparation steps and Representative examples corresponding thereto are described below. Compounds having different substituents were prepared through similar steps, but not all examples are described here. Referring to the following representative examples, those skilled in the art will be able to easily prepare compounds of Formula 1 or salts thereof having different substituents.

1-1. Preparation of Compound C According to Scheme 1

Compounds of Formula 1 according to the present invention may be prepared according to Scheme 1 as follows:

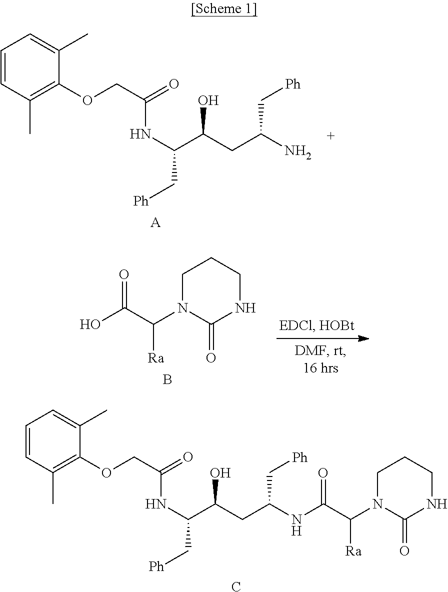

According to Scheme 1 above, Compound C was prepared from Compound A and Compound B via EDCI binding reaction. Compound C is a representative example of the compounds of Formula 1.

As an example of Compound C, (S)-N-((2S, 4S, 5S)-5-(2-(2,6-dimethylphenoxy)acetamido)-4-hydroxy-1,6-diphenylhexane-2-yl)-2-(2-oxotetrahydropyrimidin-1(2H)-yl)propanamide was obtained by using Reaction example 1 and the reaction conditions as follows:

[Reaction Example 1]

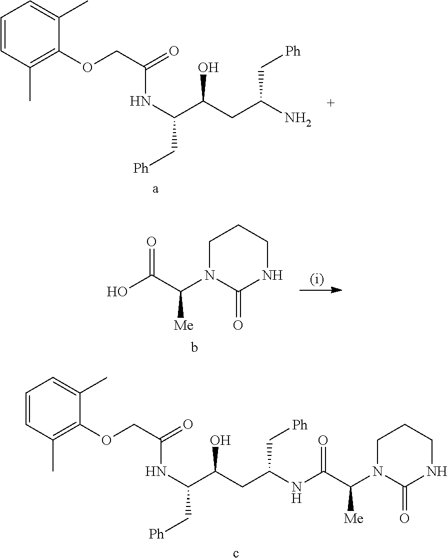

(i) Reaction conditions: after dissolving Compound a (100 mg, 0.22 mmol) in 3 mL of dimethylformamide, Compound b (43.0 mg, 0.25 mmol), 1-ethyl-3-(3-dimethylaminopropyl)carbodiimide (52 mg, 0.34 mmol) and 1-hydroxybenzotriazole (52 mg, 0.34 mmol) were added and stirred at room temperature for 16 hours. After confirming the completion of the reaction, extraction was performed using 10 mL of ethyl acetate and 20 mL of distilled water. The extracted organic layer was dried using anhydrous sodium sulfate then filtered, and the filtrate was distilled via vacuum distillation and purified using column chromatography. As a result, Compound c (72 mg, yield 54%) was obtained.

The relevant characterization data of (S)-N-((2S, 4S, 5S)-5-(2-(2,6-dimethylphenoxy)acetamido)-4-hydroxy-1,6-diphenylhexane-2-yl)-2-(2-oxotetrahydropyrimidin-1(2H)-yl)propanamide are as follows:

¹H-NMR 400 MHz (MeOD): δ 7.337.13 (10H, m), 7.02-7.00 (2H, d, J=7.2 Hz), 6.95-6.91 (1H, dd, J=8.4 Hz, J=6.4 Hz), 4.83-4.76 (1H, q, J=7.2 Hz), 4.50-4.38 (2H, m), 4.16-4.05 (2H, dd, J=27.6 Hz, J=14.8 Hz), 3.77 (1H, t, J=8.0 Hz), 3.19-3.13 (2H, m), 3.06-3.00 (1H, m), 2.98-2.87 (2H, m), 2.84-2.79 (1H, m), 2.73-2.65 (2H, m), 2.18 (6H, s), 1.72-1.70 (4H, q, J=6.8Hz), 1.17-1.15 (3H, d, J=7.2 Hz);

¹³C-NMR 175 MHz (MeOD): 5175.71, 173.37, 160.84, 158.29, 142.27, 142.24, 134.14, 133.01, 132.91, 132.53, 131.83, 129.83, 129.76, 128.23, 73.56, 72.32, 56.96, 55.95, 44.28, 44.13, 43.26, 42.91, 41.67, 25.26, 18.91, 16.65;

LRMS(ESI) calcd for C₃₅H₄₄N₄O₅ [M+H]+: 601.3, Found 601.3.

1-2. Preparation of Compound A according to Scheme 2

Compound A used as a starting material in Formula 1 can be prepared according to Scheme 2 as follows:

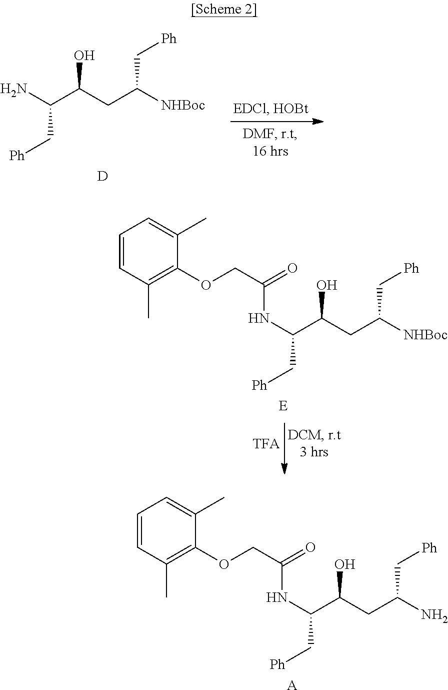

In Scheme 2, Compound E was synthesized from Compound D through a coupling reaction of (2,6-dimethylphenoxy)acetic acid and EDCI. Compound A was synthesized by deprotecting the Compound E using trifluoroacetic acid. Compound A is a representative example of a starting material used in preparing the compound of Formula 1.

As an example of Compound A, N-((2S, 4S, 55)-5-amino-3-hydroxy-1,6-diphenylhexan-2-yl)-2-(2,6-dimethylphenoxy)acetamide was obtained by using Reaction example 2 and the reaction conditions as follows:

[Reaction Example 2]

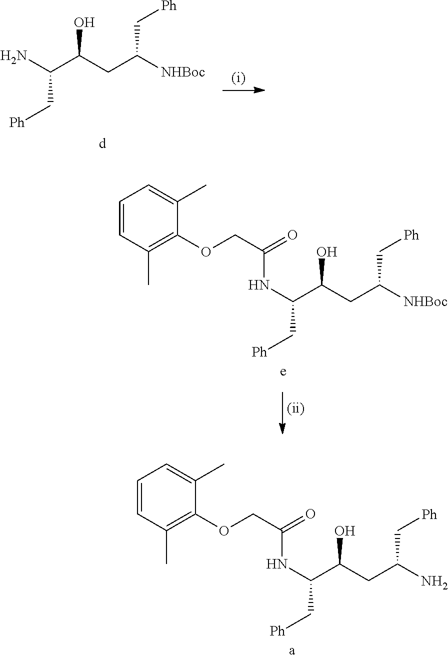

(i) Reaction conditions: Compound d (1.00 g, 2.60 mmol) was dissolved in 25 mL of dimethylformamide followed by (2,6-dimethylphenoxy)acetic acid (563 mg, 3.12 mmol), 1-ethyl-3-(3-dimethylaminopropyl) Carbodiimide (606 mg, 3.90 mmol) and 1-hydroxybenzotriazole (600 mg, 3.90 mmol) were added and stirred at room temperature for 16 hours. After confirming the completion of the reaction, extraction was performed using 100 mL of ethyl acetate and 150 mL of distilled water. The extracted organic layer was dried over anhydrous sodium sulfate, filtered, and the filtrate was distilled under reduced pressure and purified using column chromatography. As a result, Compound e (1.17 g, yield 82%) was obtained.

(ii) Reaction conditions: After dissolving Compound E in dichloromethane (1.17 g, 2.14 mmol), trifluoroacetic acid (4.92 mL, 64.2 mmol) was slowly added over 20 minutes, followed by stirring at room temperature for 3 hours. After confirming the completion of the reaction, the remaining trifluoroacetic acid and the solvent were removed by distillation under reduced pressure. Then, it was extracted using 50 mL of ethyl acetate and 50 mL of sodium bicarbonate aqueous solution. The extracted organic layer was dried over anhydrous sodium sulfate, filtered, and the filtrate was distilled under reduced pressure and purified using column chromatography. As a result, Compound A (940 mg, 98%) was obtained and was confirmed to be N-((2S, 4S, 5S)-5-amino-3-hydroxy-1,6-diphenylhexan-2-yl)-2-(2,6-dimethylphenoxy)acetamide.

1-3. Preparation of Compound B according to Scheme 3

Compound B used as a starting material in Formula 1 can be prepared according to Scheme 3 as follows:

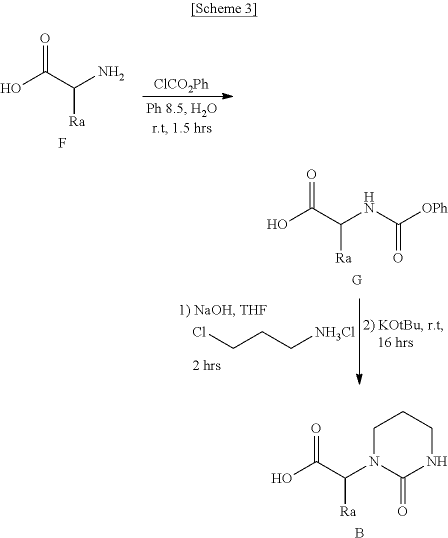

In Scheme 3, Compound G was synthesized from Compound F through Schotten-Baumann acylation using phenylchloroformate. A chloropropylurea intermediate was formed from Compound G by using 3-chloropropylamine. Compound B was prepared through a cyclization reaction frame using tert-potassium butoxideg. Compound B is a representative example of the starting material used in the preparation of the compound of Formula 1.

As an example of Compound B, (S)-2-(2-oxotetrahydropyrimidin-1(2H)-yl)propanoic acid was obtained by using Reaction example 3 and the reaction conditions as follows:

[Reaction Example 3]

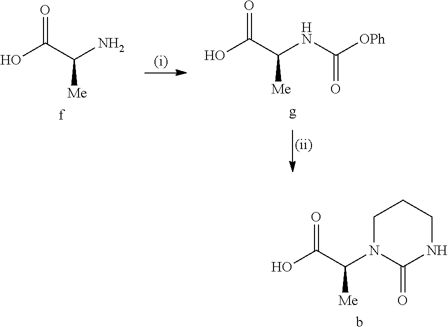

(i) Reaction conditions: Compound f (200 mg, 2.25 mmol) and sodium bicarbonate (283 mg, 3.37 mmol) were dissolved in 4 mL of distilled water, and phenylchloroformate (0.3 mL, 2.36 mmol) was added thereto, and the pH was adjusted to 8.15 to 8.6 using 50% aqueous sodium hydroxide solution. The mixture was stirred for 90 minutes, and 50% aqueous sodium hydroxide solution was periodically added to maintain the pH at 8.5 to 8.7 while stirring. After confirming the completion of the reaction, the pH was adjusted to 8.9 and dissolved solids with diethyl ether. Then, the water layer was adjusted to pH 2.0 by adding 30% aqueous sulfuric acid and extracted with diethyl ether to obtain Compound G (398 mg, yield 85%).

(ii) Reaction conditions: Compound g (300 mg, 1.67 mmol) was dissolved in 3 mL of tetrahydrofuran, and 3-chloropropylamine hydrochloride (245 mg, 1.83 mmol) and solid sodium hydroxide (202 mg, 5.05 mmol) were added, and mixed in 0° then stirred for 2 hours after raising the temperature up to 10 ° C. After confirming that the intermediate propylurea was formed, 4.20 mL of tert-potassium butoxide (1.0 M in THF) was added for 15 minutes then the mixture was stirred at room temperature for 16 hours. After confirming the completion of the reaction, the pH was adjusted to 9 using a 3N aqueous hydrochloric acid solution, and the aqueous layer was extracted. The pH of the extracted aqueous layer was adjusted to 3 then added 20 mL of ethyl acetate for extraction. The extracted organic layer was dried over anhydrous sodium sulfate, filtered, and the filtrate was distilled under reduced pressure. As a result, Compound b (102 mg, yield 41%) was obtained, and Compound b was confirmed to be (S)-2-(2-oxotetrahydropyrimidin-1(2H)-yl)propanoic acid.

In the following, characterization data including the properties, NMR data, and molecular weights of the representatively prepared compounds belonging to Formula 1 are shown below. Compounds belonging to Formula 1 with different substituents were prepared similar to the method described in Example 1.

Compound 1: (S)-N-((2S,4S,5S)-5-(2-(2,6-dimethylphenoxy)acetamido)-4-hydroxy-1,6-diphenylhexane-2-yl)-2-(2-oxotetrahydropyrimidin-1(2H)-yl)-3-phenylpropanamide

Chemical structure of Compound 1 is:

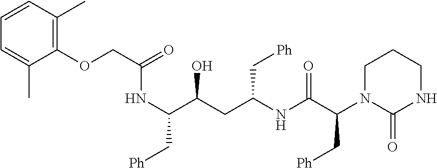 [SMILES: Cc1cccc(C)c1OCC(=O)N[C@@H](Cc1ccccc1)[C@@H](O)C[C@H](Cc1ccccc1)NC(=O)[C@H](Cc1ccccc1)N1CCCNC1=O]

Compound 1 was obtained as a white solid compound and the relevant characterization data are as follows:

¹H-NMR 400 MHz (MeOD): δ 7.367.12 (15H, m), 7.03-7.01 (2H, d, J=7.6Hz), 6.96-6.93 (1 H, dd, J=8.4Hz, J=6.4Hz), 5.05-5.01 (1 H, dd, J=7.2 Hz, J=1.2 Hz), 4.52 (1H, t, J=6.8Hz), 4.46-4.40 (1H, m), 4.18-4.02 (2H, dd, J=38.4Hz, J=14.8Hz), 3.64 (1H, t, J=6.4Hz), 3.15-3.01 (4H, m), 2.92-2.78 (4H, m), 2.73-2.64 (2H, m), 2.18 (6H, s), 1.68-1.53 (4H, m);

¹³C-NMR 175MHz (MeOD): 5174.28, 173.39, 160.74, 158.28, 142.36, 142.29, 141.44, 134.14, 130.02, 132.97, 132.61, 132.53, 131.88, 131.81, 130.00, 129.82, 129.68, 128.22, 73.53, 72.64, 61.39, 56.72, 44.76, 44.46, 43.14, 43.09, 41.74, 37.65, 25.06, 18.91;

LRMS(ESI) calcd for C₄₁H₄₈N₄O₅ [M−H]−: 675.3, Found: 675.3.

Compound 2: (S)-N-((2S,4S,5S)-5-(2-(2,6-dimethylphenoxy)acetamido)-4-hydroxy-1,6-diphenylhexane-2-yl)-2,3-dimethyl-2-(2-oxotetrahydropyrimidin-1(2H)-yl)butanamide

Chemical structure of Compound 2 is:

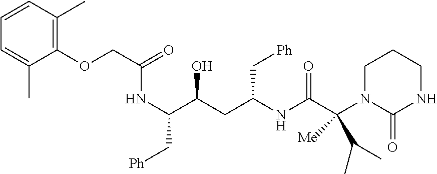 [SMILES: Cc1cccc(C)c1OCC(=O)N[C@@H](Cc1ccccc1)[C@@H](O)C[C@H](Cc1ccccc1)NC(=O)[C@](C)(C(C)C)N1CCCNC1=O]

Compound 2 was obtained as a white solid compound and the relevant characterization data are as follows:

¹H-NMR 400 MHz (MeOD): δ 7.337.13 (10H, m), 7.03-7.01 (2H, d, J=7.6 Hz), 6.97-6.93 (1H, dd, J=8.4 Hz, J=6.4 Hz), 4.40 (1H, t, J=6.4 Hz), 4.33-4.28 (1H, m), 4.13-4.02 (2H, dd, J=22.8 Hz, J=14.8 Hz), 3.85 (1H, t, J=6.0 Hz), 3.38-3.37 (1H, m), 3.31-3.21 (2H, m), 3.00-2.78 (4H, m), 2.75-2.69 (1H, dd, J=13.6 Hz, J=7.2 Hz), 2.17 (6H, s), 1.95-1.90 (2H, m), 1.74-1.62 (2H, m), 1.19 (3H, s), 0.92-0.85 (6H, dd, J=21.2Hz, J=6.4Hz);

¹³C-NMR 175 MHz (MeOD): δ 179.17, 173.23, 161.43, 158.26, 142.39, 142.36, 134.13, 133.11, 133.05, 131.79, 129.78, 129.71, 128.22, 73.51, 72.78, 70.97, 57.35, 46.29, 44.47, 43.09, 42.08, 41.61, 35.22, 25.94, 22.05, 20.80, 20.58, 18.93;

LRMS(ESI) calcd for C₃₈H₅₀N₄O₅[M−H]−: 641.3, Found : 641.3.

Compound 3: (R)-N-((2S,4S,5S)-5-(2-(2,6-dimethylphenoxy)acetamido)-4-hydroxy-1,6-diphenylhexane-2-yl)-2,3-dimethyl-2-(2-oxotetrahydropyrimidin-1(2H)-yl)butanamide

Chemical structure of Compound 3 is:

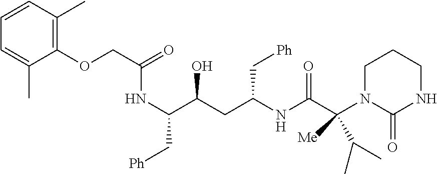 [SMILES: Cc1cccc(C)c1OCC(=O)N[C@@H](Cc1ccccc1)[C@@H](O)C[C@H](Cc1ccccc1)NC(=O)[C@@](C)(C(C)C)N1CCCNC1=O]

Compound 3 was obtained as a white solid compound and the relevant characterization data are as follows:

¹H-NMR 400 MHz (MeOD): δ 7.347.10 (10H, m), 7.03-7.01 (2H, d, J=7.6 Hz), 6.96-6.93 (1H, dd, J=8.4 Hz, J=6.4 Hz), 4.44-4.36 (2H, m), 4.13-4.09 (1H, d, J=14.8 Hz), 4.02-3.99 (1H, d, J=14.8 Hz), 3.95 (1H, t, J=6.0Hz), 3.25-3.22 (2H, m), 3.03-2.98 (1H, dd, J=13.6 Hz, J=5.6 Hz), 2.92-2.75 (4H, m), 2.17 (6H, s), 1.94-1.91 (2H, m), 1.73-1.68 (2H, m), 1.14 (3H, s), 0.91-0.84 (6H, dd, J=23.2 Hz, J=6.4 Hz);

¹³C-NMR 175 MHz (MeOD): 5179.24, 173.26, 161.63, 158.21, 142.52, 142.51, 134.14, 133.06, 132.51, 131.74, 129.73, 129.69, 128.22, 73.47, 73.26, 70.88, 57.66, 46.20, 44.61, 43.05, 42.96, 41.63, 33.53, 25.86, 21.97, 20.77, 20.54, 18.91;

LRMS(ESI) calcd for C₃₈H₅₀N₄O₅[M−H]−: 641.3, Found: 641.3.

Compound 4: N-((2S,4S,5S)-5-(2-(2,6-dimethylphenoxy)acetamido)-4-hydroxy-1,6-diphenylhexan-2-yl)-1-(2-oxotetrahydropyrimidine-1 (2H)-yl) cyclopentanecarboxamide

Chemical structure of Compound 4 is:

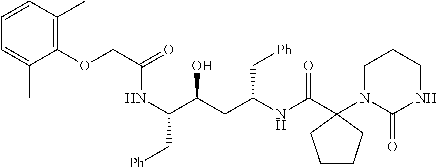 [SMILES: Cc1cccc(C)c1OCC(=O)N[C@@H](Cc1ccccc1)[C@@H](O)C[C@H](Cc1ccccc1)NC(=O)C1(N2CCCNC2=O)CCCC1]

Compound 4 was obtained as a white solid compound and the relevant characterization data are as follows:

¹H-NMR 400 MHz (MeOD): δ 7.357.14 (10H, m), 7.03-7.01 (2H, d, J=7.6 Hz), 6.97-6.93 (1H, dd, J=8.4 Hz, J=6.4 Hz), 4.51-4.47 (1H, ddd, J=9.2 Hz, J=6.0 Hz, J=1.6 Hz), 4.16-4.12 (1H, d, J=14.8 Hz), 4.06-4.02 (1H, d, J=14.8 Hz), 3.89 (1H, t, J=6.0 Hz), 3.30-3.27 (2H, m), 3.21 (2H, t, J=6.0 Hz), 3.01-2.97 (1H, dd, J=14.0 Hz, J=6.0 Hz), 2.93-2.87 (1H, dd, J=13.2 Hz, J=9.2 Hz), 2.80-2.79 (2H, d, J=7.2 Hz), 2.19 (6H, s), 2.15-2.05 (2H, m), 1.91-1.88 (2H, t, J=6.0 Hz), 1.83-1.78 (2H, m), 1.71 (2H, t, J=7.2 Hz), 1.61-1.57 (4H, m);

¹³C-NMR 175 MHz (MeOD): δ 179.45, 173.32, 161.66, 158.25, 142.44, 134.15, 133.04, 132.53, 131.75, 129.74, 128.23, 75.95, 73.50, 73.04, 57.41, 46.73, 44.58, 43.08, 43.03, 41.72, 39.43, 39.29, 27.46, 27.40, 18.90;

LRMS(ESI) calcd for C₃₅H₄₈N₄O₅[M−H]−: 639.3, Found: 639.3.

Compound 5: (S)-N-((2S,4S,5S)-5-(2-(2,6-dimethylphenoxy)acetamido)-4-hydroxy-1,6-diphenylhexane-2-yl)-2-(2-oxotetrahydropyrimidin-1(2H)-yl)-3-(m-tolyl)propanamide

Chemical structure of Compound 5 is:

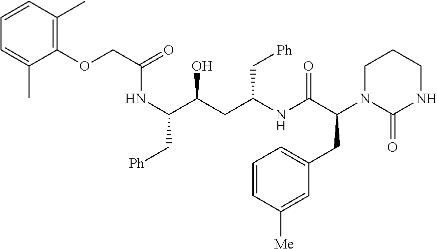 [SMILES: Cc1cccc(C[C@@H](C(=O)N[C@@H](Cc2ccccc2)C[C@H](O)[C@H](Cc2ccccc2)NC(=O)COc2c(C)cccc2C)N2CCCNC2=O)c1]

Compound 5 was obtained as a white solid compound and the relevant characterization data are as follows:

¹H-NMR 400 MHz (MeOD): δ 7.35-7.12 (11H, m), 7.07-6.93(6H, m), 5.02 (1H, t, J=8.0 Hz), 4.55-4.51 (1H, m), 4.46-4.41 (1H, m), 4.18-4.04 (2H, dd, J=39.6 Hz, J=14.8 Hz), 3.63 (1H, t, J=6.4 Hz), 3.16-3.02 (4H, m), 2.90-2.63 (6H, m), 2.27 (3H, s), 2.18 (6H, s), 1.71-1.54 (4H, m);

LRMS(ESI) calcd for C₄₂H₅₀N₄O₅ [M−H]−: 689.3, Found: 689.3.

Compound 6: (S)-N-((2S,4S,5S)-5-(2-(2,6-dimethylphenoxy)acetamido)-4-hydroxy-1,6-diphenylhexane-2-yl)-2-(2-oxotetrahydropyrimidin-1(2H)-yl)-3-(p-tolyl)propenamide

Chemical structure of Compound 6 is:

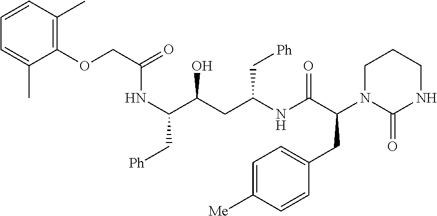 [SMILES: Cc1ccc(C[C@@H](C(=O)N[C@@H](Cc2ccccc2)C[C@H](O)[C@H](Cc2ccccc2)NC(=O)COc2c(C)cccc2C)N2CCCNC2=O)cc1]

Compound 6 was obtained as a white solid compound and the relevant characterization data are as follows:

¹H-NMR 400 MHz (MeOD): δ 7.35-7.10 (12H, m), 7.07-7.01 (4H, m), 6.97-6.93 (1H, m), 5.01 (1H, t, J=8.8 Hz), 4.53-4.49 (1H, m), 4.46-4.41 (1H, m), 4.18-4.04 (2H, dd, J=36.4 Hz, J =14.8 Hz), 3.64 (1H, t, J=7.6 Hz), 3.16-3.02 (4H, m), 2.91-2.63 (6H, m), 2.26 (3H, s), 2.18 (6H, s), 1.72-1.54 (4H, m);

LRMS(ESI) calcd for C₄₂H₅₀N₄O₅ [M−H]−: 689.3, Found: 689.3.

Compound 7: (S)-N-((2S,4S,5S)-5-(2-(2,6-dimethylphenoxy)acetamido)-4-hydroxy-1,6-diphenylhexane-2-yl)-3-(4-fluorophenyl)-2-(2-oxotetrahydropyrimidin-1(2H)-yl)propanamide

Chemical structure of Compound 7 is:

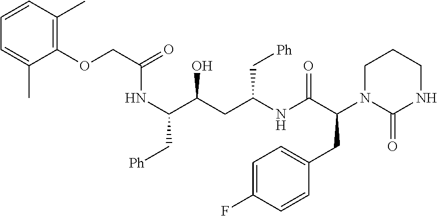 [SMILES: Cc1cccc(C)c1OCC(=O)N[C@@H](Cc1ccccc1)[C@@H](O)C[C@H](Cc1ccccc1)NC(=O)[C@H](Cc1ccc(F)cc1)N1CCCNC1=O]

Compound 7 was obtained as a white solid compound and the relevant characterization data are as follows:

¹H-NMR 400 MHz (MeOD): δ 7.36-7.15 (12H, m), 7.03-6.93 (5H, m), 5.01 (1H, t, J=8.0 Hz), 4.56-4.52 (1H, m), 4.46-4.40 (1H, m), 4.18-4.05 (2H, dd, J=38.0 Hz, J=14.8 Hz), 3.67 (1H, t, J=7.6 Hz), 3.16-3.02 (4H, m), 2.94-2.77 (4H, m), 2.71-2.64 (2H, m), 2.18 (6H, s), 1.72-1.54 (4H, m);

LRMS(ESI) calcd for C₄₁H₄₇FN₄O₅ [M−H]−: 693.3, Found: 693.3.

Compound 8: (S)-N-((2S,4S,5S)-5-(2-(2,6-dimethylphenoxy)acetamido)-4-hydroxy-1,6-diphenylhexane-2-yl)-2-(2-oxotetrahydropyrimidin-1(2H)-yl)-3-(4-(trifluoromethyl)phenyl)propanamide

Chemical structure of Compound 8 is:

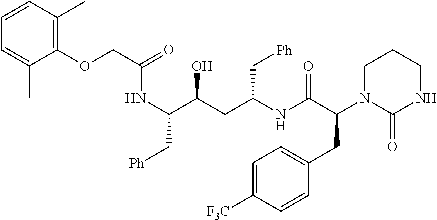 [SMILES: Cc1cccc(C)c1OCC(=O)N[C@@H](Cc1ccccc1)[C@@H](O)C[C@H](Cc1ccccc1)NC(=O)[C@H](Cc1ccc(C(F)(F)F)cc1)N1CCCNC1=O]

Compound 8 was obtained as a white solid compound and the relevant characterization data are as follows:

¹H-NMR 400 MHz (MeOD): δ 7.56-7.54 (2H, d, J=8.0 Hz), 7.45-7.43 (2H, d, J=8.0 Hz), 7.37-7.14 (10H, m), 7.03-7.01 (2H, m), 6.97-6.93 (1H, dd, J=8.4 Hz, J=6.4 Hz), 5.12-5.08 (1H, dd, J=8.8 Hz, J=6.8 Hz), 4.61-4.52 (1H, m), 4.49-4.44 (1H, m), 4.18-4.04 (2H, dd, J=36.8 Hz, J=14.8 Hz), 3.68 (1H, t, J=6.8 Hz), 3.18-3.01 (4H, m), 2.94-2.79 (4H, m), 2.68-2.62 (2H, m), 2.18 (6H, s), 1.73-1.54 (4H, m);

LRMS(ESI) calcd for C₄₂H₄₇F₃N₄O₅ [M−H]−: 743.3, Found: 743.3.

Compound 9: (S)-3-(4-cyanophenyl)-N-((2S,4S,5S)-5-(2-(2,6-dimethylphenoxy)acetamido)-4-hydroxy-1,6-diphenylhexan-2-yl)-2-(2-oxotetrahydropyrimidin-1(2H)-yl)propanamide

Chemical structure of Compound 9 is:

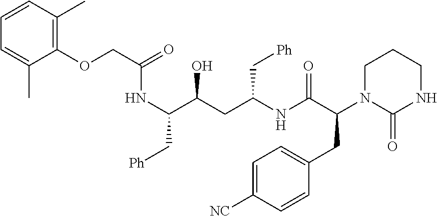 [SMILES: Cc1cccc(C)c1OCC(=O)N[C@@H](Cc1ccccc1)[C@@H](O)C[C@H](Cc1ccccc1)NC(=O)[C@H](Cc1ccc(C#N)cc1)N1CCCNC1=O]

Compound 9 was obtained as a white solid compound and the relevant characterization data are as follows:

¹H-NMR 400 MHz (MeOD): δ 7.62-7.60 (2H, d, J=8.0 Hz), 7.44-7.42 (2H, d, J=8.0 Hz), 7.36-7.16 (10H, m), 7.03-7.01 (2H, d, J=7.2 Hz), 6.98-6.93 (1H, dd, J=8.4 Hz, J=6.4 Hz), 5.11-5.07 (1 H, dd, J=8.8 Hz, J=7.2 Hz), 4.56-4.53 (1 H, m), 4.48-4.44 (1H, m), 4.21-4.06 (2H, dd, J=38.0 Hz, J=14.8 Hz), 3.66 (1H, t, J=7.6 Hz), 3.18-3.01 (4H, m), 2.93-2.80 (4H, m), 2.68-2.60 (2H, m), 2.18 (6H, s), 1.74-1.54 (4H, m);

LRMS(ESI) calcd for C₄₂H₄₇N₅O₅ [M−H]−: 700.3, Found: 700.3.

Compound 10: (S)-N-((2S,4S,5S)-5-(2-(2,6-dimethylphenoxy)acetamido)-4-hydroxy-1,6-diphenylhexane-2-yl)-2-(2-oxotetrahydropyrimidin-1(2H)-yl)-3-(thiophen-2-yl)propenamide

Chemical structure of Compound 10 is:

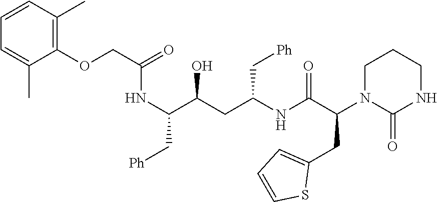 [SMILES: Cc1cccc(C)c1OCC(=O)N[C@@H](Cc1ccccc1)[C@@H](O)C[C@H](Cc1ccccc1)NC(=O)[C@H](Cc1cccs1)N1CCCNC1=O]

Compound 10 was obtained as a white solid compound and the relevant characterization data are as follows:

¹H-NMR 400 MHz (MeOD): δ 7.36-7.15 (10H, m) 7.03-7.01 (2H, d, J=7.6 Hz), 6.97-6.89 (3H, m), 4.99-4.94 (1 H, dd, J=8.8 Hz, J=7.2 Hz), 4.55-4.52 (1 H, m), 4.47-4.43 (1 H, m), 4.19-4.15 (1H, d, J=14.8 Hz), 4.10-4.06 (1H, J=14.8 Hz), 3.70 (1H, t, J=7.6 Hz), 3.30-3.24 (2H, m), 3.16-3.00 (4H, m), 2.91-2.89 (2H, d, J=7.6 Hz), 2.85-2.80 (2H, dd, J=14.0 Hz, J=5.6 Hz), 2.72-2.64 (2H, m), 2.19 (6H, s), 1.71-1.57 (4H, m);

LRMS(ESI) calcd for C₃₉H₄₆N₄O₅S [M−H]−: 681.3, Found: 681.3.

Compound 11: (S)-N-((2S,4S,5S)-5-(2-(2,6-dimethylphenoxy)acetamido)-4-hydroxy-1,6-diphenylhexane-2-yl)-2-(2-oxotetrahydropyrimidin-1(2H)-yl)-3-(thiazol-5-yl)propanamide

Chemical structure of Compound 11 is:

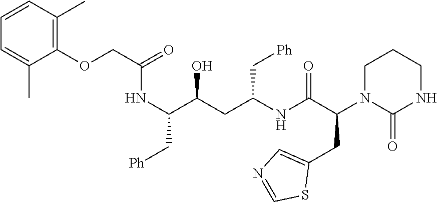 [SMILES: Cc1cccc(C)c1OCC(=O)N[C@@H](Cc1ccccc1)[C@@H](O)C[C@H](Cc1ccccc1)NC(=O)[C@H](Cc1cncs1)N1CCCNC1=O]

Compound 11 was obtained as a white solid compound and the relevant characterization data are as follows:

¹H-NMR 400 MHz (MeOD): δ 8.91 (1H, s), 7.37-7.16 (10H, m) 7.03-7.02 (2H, d, J=7.2 Hz), 6.97-6.93 (1H, dd, J=8.4 Hz, J=6.8 Hz), 5.22-5.18 (1H, dd, J=9.6 Hz, J=6.4 Hz), 4.54-4.51 (1H, m), 4.47-4.43 (1H, m), 4.19-4.15 (1H, d, J=14.8 Hz), 4.08-4.04 (1H, J=14.8 Hz), 3.75 (1 H, t, J=8.0 Hz), 3.25-3.22 (1 H, m), 3.15-3.04 (4H, m), 2.95-2.83 (3H, m), 2.73-2.66 (2H, m), 2.19 (6H, s), 1.74-1.55 (5H, m);

LRMS(ESI) calcd for C₃₈H₄₅N₅O₅S [M−H]−: 682.3, Found: 682.3.

Compound 12: (S)-3-cyclopentyl-N-((2S,4S,5S)-5-(2-(2,6-dimethylphenoxy)acetamido)-4-hydroxy-1,6-diphenylhexan-2-yl)-2-(2-oxotetrahydropyrimidin-1(2-yl)propenamide

Chemical structure of Compound 12 is:

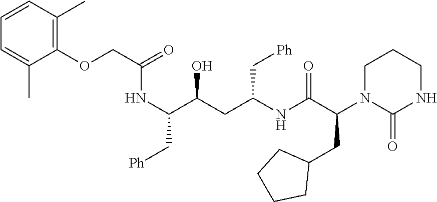 [SMILES: Cc1cccc(C)c1OCC(=O)N[C@@H](Cc1ccccc1)[C@@H](O)C[C@H](Cc1ccccc1)NC(=O)[C@H](CC1CCCC1)N1CCCNC1=O]

Compound 12 was obtained as a white solid compound and the relevant characterization data are as follows:

¹H-NMR 400 MHz (MeOD): δ 7.35-7.15 (10H, m), 7.03-7.01 (2H, d, J=7.2 Hz), 6.97-6.93 (1H, dd, J=8.4 Hz, J=6.4 Hz), 5.11-5.07 (1H, dd, J=8.8 Hz, J=7.2 Hz), 4.79-4.76 (1H, m), 4.55-4.51 (1H, m), 4.46-4.41 (1H, m), 4.18-4.07 (2H, dd, J=31.6 Hz, J=14.8 Hz), 3.80 (1H, t, J=7.2 Hz), 3.21-3.17 (2H, m), 3.07-3.01 (1H, m), 2.95-2.93 (2H, d, J=7.6 Hz), 2.86-2.81 (1H, dd, J=13.6 Hz, J=5.6 Hz), 2.78-2.65 (2H, m), 2.19 (6H, s), 1.82-1.79 (2H, m), 1.75-1.47 (12H, m), 1.20-1.12 (2H, m);

LRMS(ESI) calcd for C₄₀H₅₂N₄O₅ [M−H]−: 667.3, Found: 667.3.

Compound 13: (S)-2-cyclopentyl-N-((2S,4S,5S)-5-(2-(2,6-dimethylphenoxy)acetamido)-4-hydroxy-1,6-diphenylhexan-2-yl)-2-(2-oxotetrahydropyrimidin-1(2-yl)acetamide

Chemical structure of Compound 13 is:

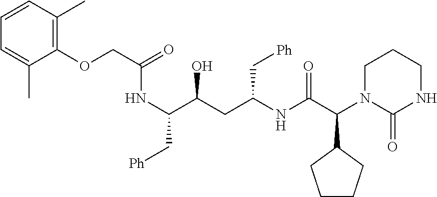 [SMILES: Cc1cccc(C)c1OCC(=O)N[C@@H](Cc1ccccc1)[C@@H](O)C[C@H](Cc1ccccc1)NC(=O)[C@H](C1CCCC1)N1CCCNC1=O]

Compound 13 was obtained as a white solid compound and the relevant characterization data are as follows:

¹H-NMR 400 MHz (MeOD): δ 7.36-7.15 (10H, m), 7.03-7.01 (2H, d, J=7.6 Hz), 6.97-6.93 (1H, dd, J=8.4 Hz, J=6.4 Hz), 4.62-4.56 (1H, m), 4.45-4.39(2H, m), 4.20-4.17 (1H, d, J=14.8 Hz), 4.09-4.05 (1H, J=14.8 Hz), 3.82 (1H, t, J=7.6 Hz), 3.20-3.08 (3H, m), 2.97-2.92 (2H, m), 2.88-2.82 (2H, m), 2.65-2.59 (2H, dd, J=13.6 Hz, J=9.2 Hz), 2.49-2.35 (1H, m), 2.20 (6H, s), 1.80-1.47 (11H, m);

LRMS(ESI) calcd for C₃₉H₅₀N₄O₅ [M−H]−: 653.3, Found: 653.3.

### Example 2

### Antiviral Activity Evaluation Test

For the host cells, 1.2×10⁴ vero cells (ATCC CCL-81) were seeded into each well of 384-well plates (black, pClear plates). Vero cells were cultured in Opti-PRO™ SFM supplemented with 4 mM L-glutamine and 1× Antibiotic-Antimycotic (Gibco/Thermo Fisher Scientific). Test compounds were added to each well immediately prior to MERS-CoV infection. The final concentration of the test compound was 10 μM, and the DMSO concentration was kept below 0.5%. The compounds prepared in Example 1 were used as test compounds, and Lopinavir, a drug for MERS-CoV, as a positive control.

Plates containing vero cells and test compounds were transferred into BL-3 medium, and infected with MERS-CoV at an MOI of 0.0625. After 24 hours, Vero cells were fixed with 4% PFA, and immunofluorescence staining was performed to detect virus-infected cells. MERS-CoV infection was observed using a Rabbit anti-MERS-CoV spike antibody, and cell viability was evaluated through Hoechst 33342 staining.

The EC50 values and CC50 values analyzed and calculated and with GraphPad Prism software are shown in Table 1 below respectively. Here, EC50 (50% efficient concentration) refers to the concentration of a compound that inhibits viral replication by 50%, and the lower the value, the better the compound can inhibit the proliferation of viruses. In addition, CC50 (50% cytotoxicity concentration) refers to the concentration of a compound that kills 50% of the host cells, and higher the value, the lower the toxicity of the compound to the host cells.

The SI value was calculated by dividing the calculated CC50 value by the EC50 value. SI (selectivity index) indicates the relative effectiveness of a specific compound, and a higher value means that it exhibits maximum antiviral activity with minimum cytotoxicity.

TABLE 1

| | Compounds | EC50 | CC50 | SI |
|---|---|---|---|---|
| Positive control | 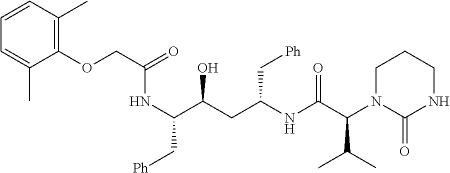 [SMILES: Cc1cccc(C)c1OCC(=O)N[C@@H](Cc1ccccc1)[C@@H](O)C[C@H](Cc1ccccc1)NC(=O)[C@H](C(C)C)N1CCCNC1=O] | 10.47 | >50 | 4.78 |
| 1 | 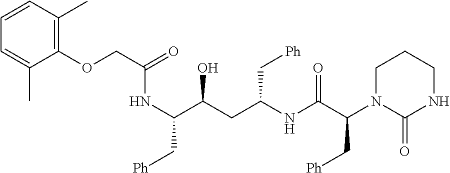 [SMILES: Cc1cccc(C)c1OCC(=O)N[C@@H](Cc1ccccc1)[C@@H](O)C[C@H](Cc1ccccc1)NC(=O)[C@H](Cc1ccccc1)N1CCCNC1=O] | 2.03 | >50 | 21.74 |
| 2 | 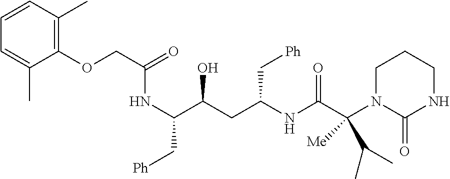 [SMILES: Cc1cccc(C)c1OCC(=O)N[C@@H](Cc1ccccc1)[C@@H](O)C[C@H](Cc1ccccc1)NC(=O)[C@](C)(C(C)C)N1CCCNC1=O] | N/D | >50 | N/D |
| 3 | 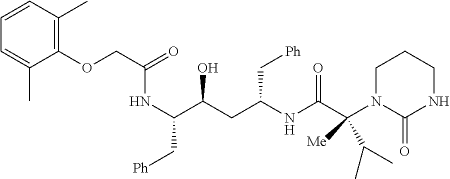 [SMILES: Cc1cccc(C)c1OCC(=O)N[C@@H](Cc1ccccc1)[C@@H](O)C[C@H](Cc1ccccc1)NC(=O)[C@@](C)(C(C)C)N1CCCNC1=O] | N/D | >50 | N/D |
| 4 | 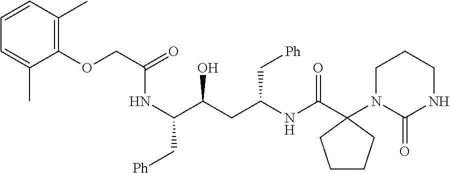 [SMILES: Cc1cccc(C)c1OCC(=O)N[C@@H](Cc1ccccc1)[C@@H](O)C[C@H](Cc1ccccc1)NC(=O)C1(N2CCCNC2=O)CCCC1] | 7.66 | >50 | 6.53 |
| 5 | 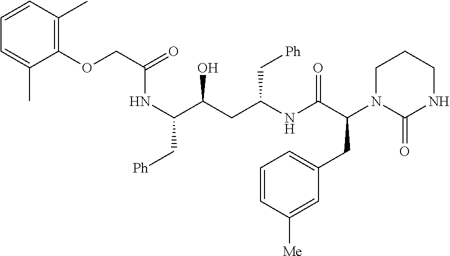 [SMILES: Cc1cccc(C[C@@H](C(=O)N[C@@H](Cc2ccccc2)C[C@H](O)[C@H](Cc2ccccc2)NC(=O)COc2c(C)cccc2C)N2CCCNC2=O)c1] | 2.79 | >50 | 17.91 |
| 6 | 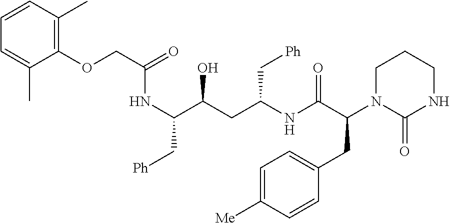 [SMILES: Cc1ccc(C[C@@H](C(=O)N[C@@H](Cc2ccccc2)C[C@H](O)[C@H](Cc2ccccc2)NC(=O)COc2c(C)cccc2C)N2CCCNC2=O)cc1] | 2.06 | >50 | 24.26 |
| 7 | 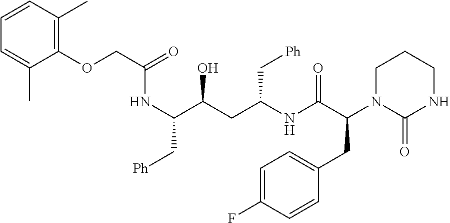 [SMILES: Cc1cccc(C)c1OCC(=O)N[C@@H](Cc1ccccc1)[C@@H](O)C[C@H](Cc1ccccc1)NC(=O)[C@H](Cc1ccc(F)cc1)N1CCCNC1=O] | 2.09 | >50 | 23.96 |
| 8 | 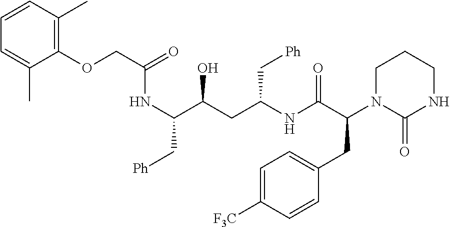 [SMILES: Cc1cccc(C)c1OCC(=O)N[C@@H](Cc1ccccc1)[C@@H](O)C[C@H](Cc1ccccc1)NC(=O)[C@H](Cc1ccc(C(F)(F)F)cc1)N1CCCNC1=O] | 1.22 | >50 | 40.88 |
| 9 | 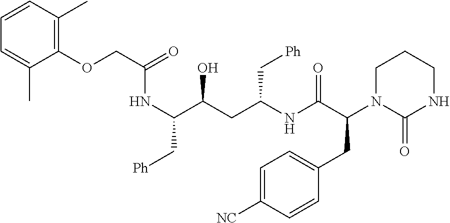 [SMILES: Cc1cccc(C)c1OCC(=O)N[C@@H](Cc1ccccc1)[C@@H](O)C[C@H](Cc1ccccc1)NC(=O)[C@H](Cc1ccc(C#N)cc1)N1CCCNC1=O] | 2.59 | >50 | 19.33 |
| 10 | 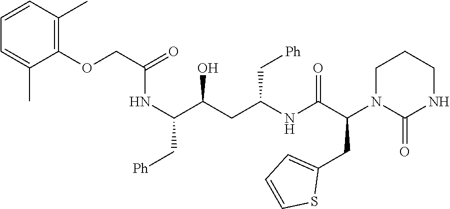 [SMILES: Cc1cccc(C)c1OCC(=O)N[C@@H](Cc1ccccc1)[C@@H](O)C[C@H](Cc1ccccc1)NC(=O)[C@H](Cc1cccs1)N1CCCNC1=O] | 3.32 | >50 | 15.06 |

As shown in Table 1 above, Compounds 1 to 10 exhibit effective inhibitory activity against viral proliferation and replication of MERS-CoV while exhibiting minimal toxicity to host cells. In particular, it has been demonstrated that the compounds according to the present invention have higher safety and efficacy compared to the positive control, Lopinavir. This shows that the pharmaceutical composition comprising the compound according to the present invention or a pharmaceutically acceptable salt thereof can be used as a standard treatment for Middle East Respiratory Syndrome, particularly as a single treatment.

1. A compound represented by Formula 1 or a pharmaceutically acceptable salt thereof: 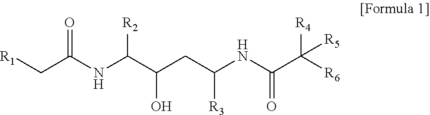
Wherein,
R₁ is —OR₇ or —SR₇,
R₇ is C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl or C3-10 heterocycloalkylalkyl, substituted or unsubstituted by one or more substituent selected among halogen, C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl and C3-10 heterocycloalkylalkyl;
R₂ and R₃ are each independently a hydrogen atom, C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl, or C3-10 heterocycloalkylalkyl;
R₅ is 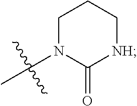 [SMILES: *N1CCCNC1=O]
in case that R₄ is a hydrogen atom,
R₆ is C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl or C3-10 heterocycloalkylalkyl, substituted or unsubstituted by one or more substituent selected among halogen, C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl, C3-10 heterocycloalkylalkyl, —CF₃ and —CN;
in case that R₄ is C1-C6 alkyl,
R₆ is C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl, or C3-10 heterocycloalkylalkyl;
R₄ and R₆ can combine with each other to form C6-C12 aryl, C3-C12 cycloalkyl, C3-10 heteroaryl or C3-10 heterocycloalkyl.

2. The compound or pharmaceutically acceptable salt thereof according to claim 1,
wherein R₂ and R₃ are each independently a C6-C12 aryl, C6-C12 arylalkyl, C3-10 heteroaryl or C3-10 heteroarylalkyl.

3. The compound or pharmaceutically acceptable salt thereof according to claim 1,
wherein R₄ is a hydrogen atom, and
R₆ is C6-C12 aryl, C6-C12 arylalkyl, C3-10 heteroaryl or C3-10 heteroarylalkyl, substituted or unsubstituted by one or more substituent selected among halogen, C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl, C3-10 heterocycloalkylalkyl, —CF₃ and —CN.

4. The compound or pharmaceutically acceptable salt thereof according to claim 1,
wherein R₄ is C1-C6 alkyl, and
R₆ is C1-C6 alkyl, C2-C6 alkenyl, C2-C6 alkynyl, C6-C12 aryl, C6-C12 arylalkyl, C3-C12 cycloalkyl, C3-C12 cycloalkylalkyl, C3-10 heteroaryl, C3-10 heteroarylalkyl, C3-10 heterocycloalkyl, or C3-10 heterocycloalkylalkyl.

5. The compound or pharmaceutically acceptable salt thereof according to claim 1,
wherein the compound represented by Formula 1 is selected from the group consisting of:

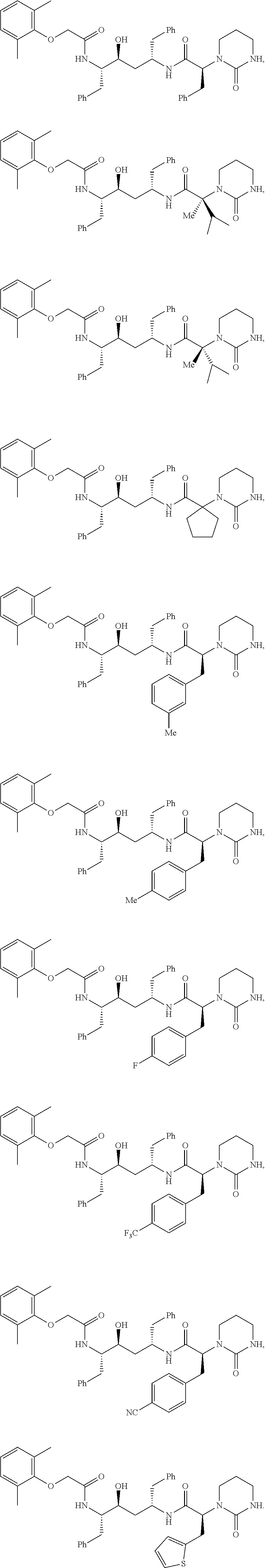

6. A pharmaceutical composition for treating or preventing Middle East Respiratory Syndrome, comprising the compound or pharmaceutically acceptable salt thereof according to claim 1.

7. The pharmaceutical composition according to claim 6, wherein it is administered simultaneously or sequentially with one or more antiviral agents.
